(19) United States
(12) Patent Application Publication
(10) Pub. No.: US 2016/0296189 A1
(43) Pub. Date: Oct. 13, 2016

(54) RADIOGRAPHIC IMAGE CAPTURING SYSTEM

(71) Applicant: Konica Minolta, Inc., Tokyo (JP)

(72) Inventors: Kenichirou SUZUKI, Tokyo (JP); Koji TAKEMURA, Tokyo (JP)

(21) Appl. No.: 15/091,923

(22) Filed: Apr. 6, 2016

(30) Foreign Application Priority Data
Apr. 7, 2015 (JP) 2015-078113

(51) Int. Cl. A61B 6/00

(52) U.S. Cl. CPC A61B 6/5241

(57) ABSTRACT

A radiographic image capturing system includes the following. A capturing stand includes a holder which can hold a plurality of radiographic image capturing devices. A radiation irradiator is able to apply radiation to the radiographic image capturing devices loaded in the holder at once. A console carries out image processing on image data acquired by the radiographic image capturing devices. The console carries out the image processing through application of a parameter applied to image data acquired by a radiographic image capturing device assigned with a predetermined number to image data acquired by the other radiographic image capturing devices. The predetermined number is a number from multiple numbers assigned to the radiographic image capturing devices loaded in the holder in an ascending order from a head to toe of a patient.

### CROSS REFERENCE TO RELATED APPLICATIONS

The present invention claims priority under 35 U.S.C. §119 to Japanese Application No. 2015-078113 filed Apr. 7, 2015, the entire content of which is incorporated herein by reference.

### BACKGROUND OF THE INVENTION

1. Field of the Invention

The present invention relates to a radiographic image capturing system, specifically, a radiographic image capturing system including a capturing stand for capturing a long-length image (stitch image) by one-shot exposure.

2. Description of Related Art

Conventional radiographic image capturing devices (flat panel detectors) F capture long radiographic images of relatively large areas of patients, e.g., full-spine images or full-leg images. As illustrated in FIG. 20A, such a radiographic image capturing device F is loaded in a holder 101 and movable along a support 102 of a capturing stand 100. The position of the image capturing device F is changed in the body axis A direction (i.e., vertical direction) of a patient P as a subject while irradiating with radiation emitted from a radiation irradiator 103 to capture multiple radiographic images for capturing a long-length image.

With reference to FIG. 20A, a collimator 104 having an aperture (not shown) is disposed between the radiation irradiator 103 and the radiographic image capturing device F and moved in the vertical direction in synchronization with the radiographic image capturing device F to narrow a radiation irradiated field. The multiple radiographic images captured as described above are combined at a console (not shown) into a single long-length image (for example, refer to Japanese Patent Application Laid-Open Publication No. 2013-154146).

Unfortunately, the patient P as the subject moves during capturing of the multiple radiographic images through multiple exposures to radiation emitted from the radiation irradiator 103 while the radiographic image capturing device F is moved to different positions, as illustrated in FIG. 20A. This causes the problem of body movement. If the patient P moves during capturing of any one of the multiple radiographic images, an appropriate long-length image cannot be readily acquired even through recapturing of the relevant radiographic image and combining it with the other radiographic images. As a result, all of the multiple radiographic images need to be recaptured (in other words, the entire long-length image needs to be recaptured). This leads to an increase in radiation dose to which the patient P is exposed.

With reference to FIG. 20B, a capturing stand 200 is developed that includes a holder 201 carrying multiple radiographic image capturing devices F1 to F3 aligned along the body axis A of the patient P (for example, refer to Japanese Patent Application Laid-Open Publication No. 2012-045159). A capturing stand having such a configuration can capture multiple radiographic images through a single exposure (one-shot exposure) of radiation emitted from the radiation irradiator 103 without movement of the aligned radiographic image capturing devices F1 to F3 along the body axis A of the patient P. Thus, the movement of the patient P does not cause the problem described above.

A capturing stand including a holder that can carry multiple radiographic image capturing devices F, such as that illustrated in FIG. 20B, can capture a long-length image through a single exposure of radiation to the multiple radiographic image capturing devices F loaded in the holder and is referred to as “capturing stand for capturing a long-length image by one-shot exposure.” Besides the vertical capturing stand 200 illustrated in FIG. 20B, a horizontal capturing stand 300, such as that illustrated in FIG. 21, may also be used to capture a long-length image by one-shot exposure. The capturing stand 300 includes a holder 301 horizontally disposed below a top panel 302 and carrying multiple radiographic image capturing devices F1 to F3. The holder 301 is movable along the horizontal direction to capture a patient P as a subject laying or sitting on the top panel 302.

The holder of a capturing stand for capturing a long-length image by one-shot exposure, such as that illustrated in FIG. 20B or 21, can carry three radiographic image capturing devices F1 to F3. For example, all three radiographic image capturing devices F1 to F3 are often loaded in the holder to capture a front image of a full leg of an adult patient P, whereas a front image of a full leg of a young patient P, i.e., a child or infant, can be captured with only two radiographic image capturing devices F loaded in the holder.

A difference in quality of captured long-length images “plong” has become apparent between a case of capturing of a front face full-leg image with two radiographic image capturing devices F1 and F2 and a case of capturing of a front face full-leg image with two radiographic image capturing devices F2 and F3, with the devices F1 to F3 being loaded in the holder 301 of the capturing stand 300 illustrated in FIG. 21.

For example, the holder 301 of the capturing stand 300 illustrated in FIG. 21 has three loading positions placed from the head to toe of the patient P as the subject. The two radiographic image capturing devices F1 and F2 are loaded in the holder 301 at the two loading positions closer to the head of the patient P among the three loading positions in the holder 301 (the loading positions at which the radiographic image capturing devices F1 and F2 are loaded in FIG. 21). The front long-length image of a full leg is captured by positioning the holder 301 carrying the two radiographic image capturing devices F1 and F2 below the leg of the patient P who is an infant, as illustrated in FIG. 22A, to give a long-length image “plong”, such as that illustrated in FIG. 23A.

The two radiographic image capturing devices F2 and F3 are loaded in the holder 301 of the capturing stand 300 at the two loading positions closer to the toe of the patient P among the three loading positions (the loading positions at which the radiographic image capturing devices F2 and F3 are loaded in FIG. 21). The front long-length image of a full leg is captured by positioning the holder 301 carrying the two radiographic image capturing devices F2 and F3 below the leg of the patient P who is an infant, as illustrated in FIG. 22B to give a long-length image “plong,” such as that illustrated in FIG. 23B.

It has been discovered that the long-length image “plong” illustrated in FIG. 23A (i.e., a long-length image “plong” captured with the configuration illustrated in FIG. 22A) has overall brightness and contrast higher than those of the long-length image “plong” illustrated in FIG. 23B (i.e., a long-length image “plong” captured with the configuration illustrated in FIG. 22B). In other words, it has been discovered that the two long-length images have different image qualities. This phenomenon also occurs in images captured with a vertical capturing stand for capturing a long-length image by one-shot exposure (for example, the capturing stand illustrated in FIG. 20B).

Radiographic image capturing devices F loaded at different positions in the holder of a capturing stand capture long-length images “plong” with different image qualities. Thus, the image quality of the long-length images “plong” varies depending on the loading positions of the radiographic image capturing devices F relative to the holder of the capturing stand. Such difference in image quality precludes comparison of the long-length images “plong” through observation by a medical doctor, for example.

A desirable radiographic image capturing system that captures a long-length image by one-shot exposure should generate long-length images “plong” having substantially the same image qualities regardless of the loading positions of the radiographic image capturing devices F in the holder of the capturing stand.

### BRIEF SUMMARY OF THE INVENTION

An object of the present invention, which has been conceived in light of the problems described above, is to provide a radiographic image capturing system that can generate one-shot long-length images having substantially the same image qualities regardless of the loading positions of radiographic image capturing devices in a holder of a capturing stand.

According to an aspect of the present invention there is provided a radiographic image capturing system including: a capturing stand including a holder which can hold a plurality of radiographic image capturing devices; a radiation irradiator that is able to apply radiation to the radiographic image capturing devices loaded in the holder at once; and a console that carries out image processing on image data acquired by the radiographic image capturing devices, wherein, the console carries out the image processing during capturing of a long-length image acquired by the radiographic image capturing devices loaded in the holder of the capturing stand through application of a parameter applied to image data acquired by a radiographic image capturing device assigned with a predetermined number to image data acquired by the other radiographic image capturing devices, the predetermined number being a number from multiple numbers assigned to the radiographic image capturing devices loaded in the holder during the application of radiation from the radiation irradiator in an ascending order from a head to toe of a patient as a subject, a number of the radiographic image capturing devices loaded in the holder being smaller or equal to a maximum number of radiographic image capturing devices loadable in the holder.

According to the present invention, it is possible to provide a radiographic image capturing system that can generate one-shot long-length images having substantially the same image qualities regardless of the loading positions of radiographic image capturing devices in a holder of a capturing stand.

### BRIEF DESCRIPTION OF THE DRAWINGS

The present invention will become more fully understood from the detailed description given hereinbelow and the appended drawings, and thus are not intended to define the limits of the present invention, and wherein;

FIG. 1 illustrates the configuration of a radiographic image capturing system according to an embodiment;

FIG. 2 illustrates an example configuration of a radiographic image capturing system including multiple capturing rooms linked to at least one console;

FIG. 3 illustrates another example configuration of a capturing stand for capturing a long-length image by one-shot exposure;

FIG. 4A illustrates an exposure switch for a radiation irradiator;

FIG. 4B illustrates the exposure switch pushed halfway;

FIG. 4C illustrates the exposure switch fully pushed;

FIG. 5 is a perspective view illustrating the exterior of a radiographic image capturing device;

FIG. 6 is a block diagram illustrating the equivalent circuit of a radiographic image capturing device;

FIG. 7A illustrates dongles disposed at loading positions on a holder of a capturing stand;

FIG. 7B illustrates a dongle in connection with a connector of a radiographic image capturing device;

FIG. 8 is a table showing an example of capturing order information;

FIG. 9 is a table showing an example selection menu for selecting items of the capturing order information;

FIG. 10 illustrates an example menu screen displayed on a display unit of a console;

FIG. 11 is a timing chart illustrating the timing of application of an ON voltage to scanning lines during a resetting process of radiation detectors, a charge accumulation mode, and a reading process of image data;

FIG. 12 is a timing chart illustrating the timing of application of an ON voltage to scanning lines until completion of a reading process of offset data;

FIG. 13 illustrates a preview image on a menu screen;

FIG. 14A illustrates example images captured by the radiographic image capturing devices;

FIG. 14B illustrates the combining of the images;

FIG. 15 illustrates an example long-length image generated through combination of images;

FIG. 16 illustrates the long-length image generated through combination of images on the menu screen;

FIG. 17A illustrates capturing of a long-length image of the full front view of a leg with radiographic image capturing devices loaded in two positions closer to the head of a patient among the loading positions in the holder;

FIG. 17B illustrates capturing of a long-length image of a full front view of a leg with radiographic image capturing devices loaded in two positions closer to the toe of the patient among the loading positions in the holder;

FIG. 18A illustrates an example long-length image generated with radiographic image capturing devices loaded in the radiographic image capturing system according to an embodiment, as illustrated in FIG. 17A;

FIG. 18B illustrates an example long-length image generated with radiographic image capturing devices loaded in the radiographic image capturing system according to an embodiment, as illustrated in FIG. 17B;

FIG. 19 illustrates preview images generated in accordance with this embodiment and displayed on the main menu on a menu screen as wipe images;

FIG. 20A illustrates conventional capturing of long-length images;

FIG. 20B illustrates capturing of a long-length image by one-shot exposure;

FIG. 21 illustrates a horizontal capturing stand for capturing a long-length image by one-shot exposure;

FIG. 22A illustrates capturing of a long-length image of a full front view of a leg with radiographic image capturing devices loaded in two positions closer to the head of a patient among the loading positions in the holder;

FIG. 22B illustrates capturing of a long-length image of a full front view of a leg with radiographic image capturing devices loaded in two positions closer to the toe of the patient among the loading positions in the holder;

FIG. 23A illustrates an example long-length image generated with radiographic image capturing devices, loaded as those illustrated in FIG. 22A, in a traditional system; and

FIG. 23B illustrates an example long-length image generated with radiographic image capturing devices, loaded as those illustrated in FIG. 22B, in a traditional system.

### DETAILED DESCRIPTION OF PREFERRED EMBODIMENTS

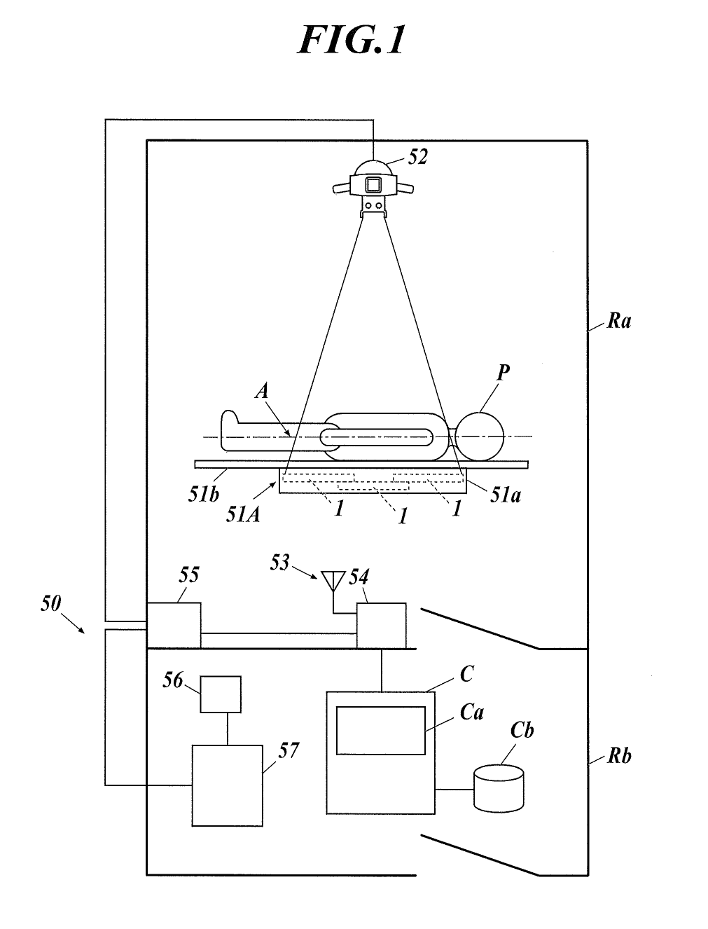

A radiographic image capturing system according to an embodiment of the present invention will now be described with reference to the accompanying drawings. FIG. 1 illustrates the configuration of a radiographic image capturing system according to this embodiment.

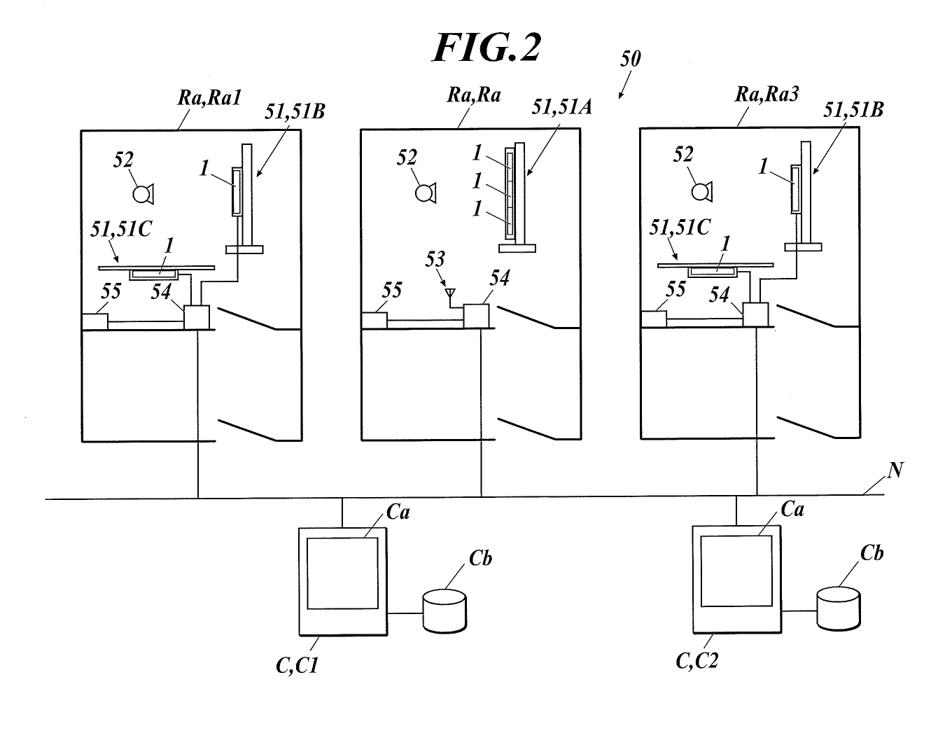

FIG. 1 illustrates a capturing room Ra containing only a capturing stand 51A for capturing a long-length image by one-shot exposure. The capturing room Ra may also contain other capturing stands, such as a vertical capturing stand 51B and a horizontal capturing stand 51C for plain radiographic capturing (see FIG. 2). That is, when there is only one capturing room Ra, the capturing stand 51A for capturing a long-length image by one-shot exposure should be installed in the capturing room Ra and any other additional modalities may be optionally installed in the capturing room Ra.

The basic configuration of a radiographic image capturing system 50 according to this embodiment is illustrated in FIG. 1 in which the capturing room Ra and a console C are connected to establish a 1:1 relationship. Alternatively, multiple capturing rooms Ra (Ra1 to Ra3) may be connected to at least one console C (C1 and C2) via a network N, as illustrated in FIG. 2.

If there are multiple capturing rooms Ra as illustrated in FIG. 2, at least one of these capturing rooms Ra should be provided with a capturing stand 51A for capturing a long-length image by one-shot exposure, and any other additional modalities may be optionally installed in the capturing room Ra containing the capturing stand 51A and the other capturing rooms Ra. Alternatively, capturing stands 51A for capturing long-length image by one-shot exposure may be installed in all the capturing rooms Ra.

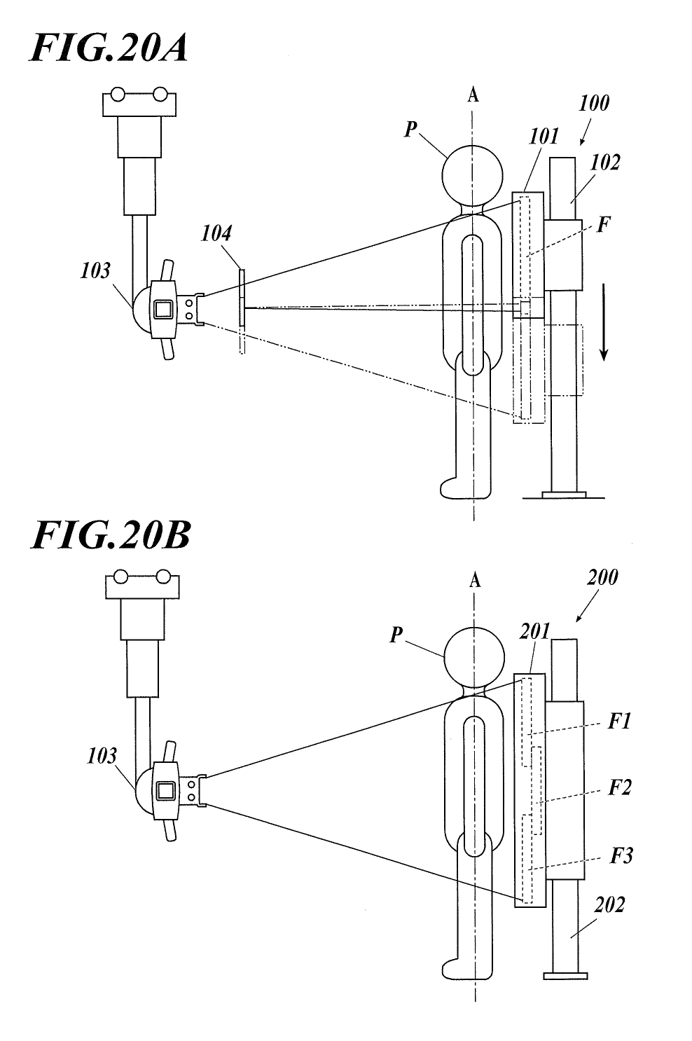

Hereinafter, the capturing stand 51A for capturing a long-length image by one-shot exposure may also be simply referred to as “capturing stand 51A.” FIG. 1 illustrates recumbent image capturing of a patient P as a subject laying or sitting on a top panel 51b of the capturing stand 51A for capturing a long-length image by one-shot exposure. Alternatively, the capturing stand 51A for capturing a long-length image by one-shot exposure according to the present invention may be applied to upright image capturing of a patient P standing in front of a holder loaded with multiple radiographic image capturing devices, as illustrated in FIGS. 2 and 20B.

### [Basic Configuration of Radiographic Image Capturing System]

With reference to FIG. 1, the capturing room Ra (or at least one of the multiple capturing rooms Ra (see FIG. 2)) according to this embodiment contains a capturing stand 51A for capturing a long-length image in a single exposure, which can hold multiple radiographic image capturing devices 1 for capturing a long-length image. The capturing stand 51A includes a holder 51a that can carry multiple radiographic image capturing devices 1 aligned along the body axis A of a patient P as a subject. The holder 51a is horizontally disposed below a top panel 51b and is movable along the horizontal direction for positioning.

With reference to FIGS. 1 and 2, loading of three radiographic image capturing devices 1 in the holder 51a of the capturing stand 51A will now be described. Alternative to three radiographic image capturing devices 1, four or more radiographic image capturing devices 1 may be loaded in the capturing stand 51A in the present invention.

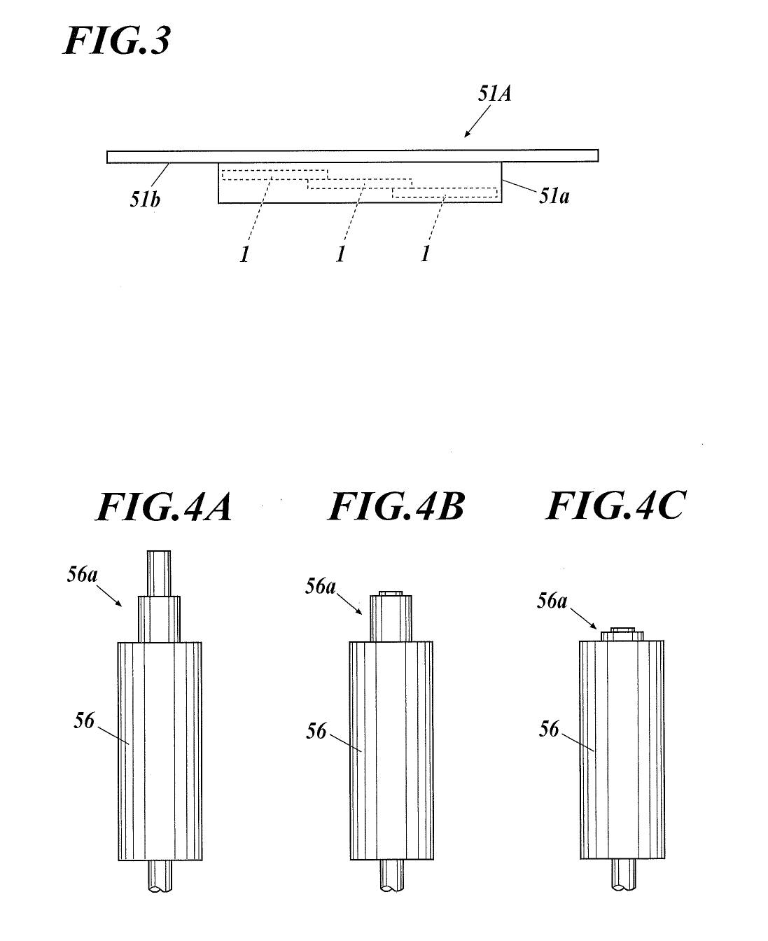

With reference to FIG. 1, multiple radiographic image capturing devices 1 are staggered in the holder 51a so as to be alternately adjacent to or remote from a radiation irradiator 52. Alternatively, as illustrated in FIG. 3, the radiographic image capturing devices 1 are disposed from one end (the right end in FIG. 3) of the holder 51a to the other end (the left end in FIG. 3) toward the radiation irradiator 52 (not shown) disposed at the top of FIG. 3.

The capturing room Ra contains the radiation irradiator 52. With reference to FIG. 1, the radiation irradiator 52 for capturing a long-length image is of a wide-angle radiation type that can simultaneously expose the multiple radiographic image capturing devices 1 loaded in the capturing stand 51A through a single exposure (one-shot exposure) of the patient P as the subject with radiation. The radiation irradiator 52 may also be used for both simple vertical and horizontal capturing. To perform simple capturing, the field irradiated with radiation emitted from the radiation irradiator for capturing long-length images can be narrowed with a collimator.

The capturing room Ra is provided with a relay 54 for relaying the communication between units inside the capturing room Ra and units outside the capturing room Ra. The relay 54 includes an access point 53 so that the radiographic image capturing devices 1 can wirelessly transmit and receive image data D and other signals. In FIGS. 1 and 2, each radiographic image capturing device 1, which is loaded in the holder 51a of the capturing stand 51A, is wirelessly connected to the relay 54. Alternatively, the capturing stand 51A, the radiographic image capturing devices 1, and the relays 54 may be connected via communication lines.

The relay 54 is connected to a controller 55 of the radiation irradiator 52 and the console C. The relay 54 includes a converter (not shown) that converts signals for a local area network (LAN) communication to be sent from the radiographic image capturing devices 1 or the console C to the controller 55 of the radiation irradiator 52 into signals for the controller 55, or vice versa.

A console 57 of the radiation irradiator 52 is installed in a front chamber (operating chamber) Rb, as illustrated in FIG. 1. The console 57 includes an exposure switch 56 to be operated by an operator or radiologist to instruct the start of radiation to the radiation irradiator 52. The console 57 can be operated to instruct the controller 55 of the radiation irradiator 52 to determine tube voltage, tube current, irradiation time, and other parameters. The console C may also be configured to instruct such determination and modification of the tube voltage and other parameters.

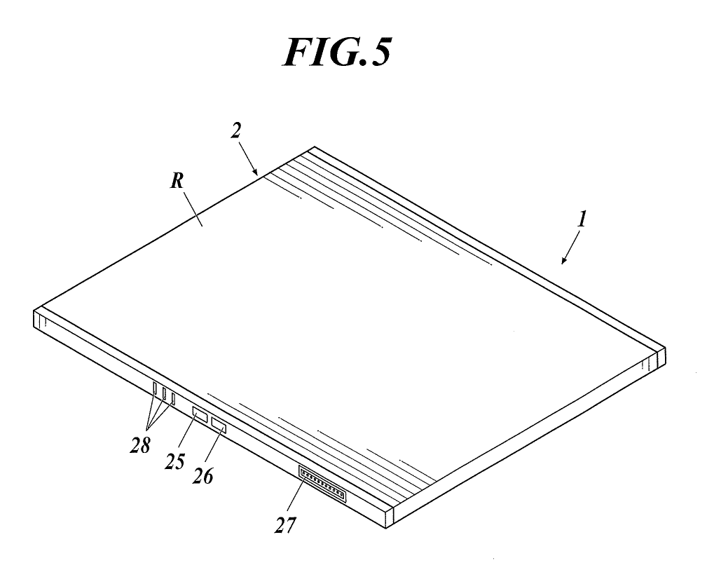

With reference to FIG. 4A, the exposure switch 56 includes a button 56a. The first operation of the button 56a of the exposure switch 56 is performed (the button is pressed halfway), as illustrated in FIG. 4B, by an operator or radiologist to instruct the controller 55 to start the radiation irradiator 52. Then, the second operation of the button 56a of the exposure switch 56 is operated (the button is fully pressed), as illustrated in FIG. 4C, by the operator to instruct the controller 55 to instruct the radiation irradiator 52 to emit radiation. The emission of radiation from the radiation irradiator 52 will be described below.

The front chamber Rb is provided with the console C that is constituted of a computer (not shown) including a central processing unit (CPU), a read only memory (ROM), a random access memory (RAM), and an input/output interface, connected to each other via a bus. The radiographic image capturing system. 50 having the configuration illustrated in FIG. 2 may include a console C disposed outside the capturing room.

The console C includes a display unit Ca including a cathode ray tube (CRT) or a liquid crystal display (LCD), and an input unit including a mouse and a keyboard (not shown). The console C is connected to an external or internal storage unit Cb including a hard disk drive (HDD).

Although not illustrated, the console C is connected to a hospital information system (HIS), a radiology information system (RIS), a picture archiving and communication system (PACS), and/or a quality assurance (QA) station via a network N.

### [Radiographic Image Capturing Devices]

The radiographic image capturing devices 1 used in the radiographic image capturing system will now be described. FIG. 5 is a perspective view illustrating the exterior of a radiographic image capturing device.

The radiographic image capturing devices 1 according to this embodiment each includes a casing 2 accommodating radiation detectors 7 and other components described below. One of the side faces of the casing 2 is provided with a power switch 25, a selector switch 26, the connector 27 mentioned above, and indicators 28.

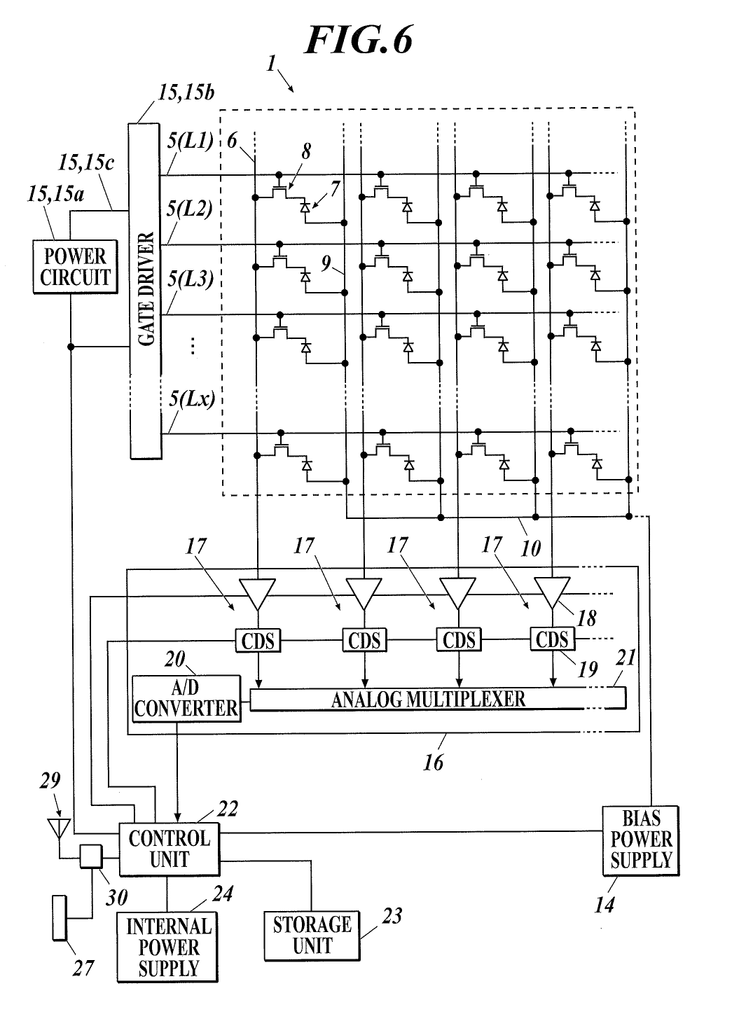

Although not illustrated, the opposite side face of the casing 2 according to this embodiment is provided with an antenna 29 (see FIG. 6) for wireless communication with external units. A cable (not shown) can be connected to the connector 27 to establish wire communication with an external unit.

FIG. 6 is a block diagram illustrating the equivalent circuit of a radiographic image capturing device. With reference to FIG. 6, multiple radiation detectors 7 are disposed in a two-dimensional array or matrix on a sensor substrate (not shown) of a radiographic image capturing device 1. The radiation detectors 7 each generate an electrical charge depending on the intensity of emitted radiation. The radiation detectors 7 are connected to respective bias lines 9, which are connected to respective connecting lines 10. The connecting lines 10 are connected to a bias power supply 14. The bias power supply 14 applies an inverse bias voltage to the radiation detectors 7 via the bias lines 9.

The radiation detectors 7 are connected to thin film transistors (TFTs) 8, which serve as switching devices and are connected to respective signal lines 6. In a scan driver 15, a power circuit 15a supplies ON and OFF voltages to a gate driver 15b via a line 15c. The gate driver 15b switches the ON and OFF voltages applied to lines L1 to Lx of scanning lines 5. The TFTs 8 are turned on in response to an ON voltage applied via the scanning lines 5 and cause the electrical charge accumulated in the radiation detectors 7 to be discharged via the signal lines 6. The TFTs 8 are turned off in response to an OFF voltage applied via the scanning lines 5 to disconnect the radiation detectors 7 and the respective signal lines 6 and cause accumulation of the electrical charges in the radiation detectors 7.

Multiple reader circuits 17 are provided in a reader IC 16 and connected to the respective signal lines 6. During the reading process of image data D, electrical charges discharged from the radiation detectors 7 flow into the reader circuits 17 via the signal lines 6, and voltage values corresponding to the electrical charges are output from amplifier circuits 18. Correlated double sampling circuits (“CDSs” in FIG. 6) 19 read the voltage values from the amplifier circuits 18 and output analog image data items D corresponding to the voltage values to the components downstream.

The image data items D are sequentially sent to an A/D converter 20 via an analog multiplexer 21, converted to digital image data items D at the A/D converter 20, and then output to and stored in a storage unit 23.

A control unit 22 includes a computer (not shown) provided with a CPU, a ROM, a RAM, and an input/output interface connected to a bus, and a field programmable gate array (FPGA). The control unit 22 may be composed of a dedicated controller circuit. The control unit 22 is connected to the storage unit 23 provided with a static RAM (SRAM), a synchronous DRAM (SDRAM), and a NAND flash memory.

The control unit 22 is connected to a communication unit 30 that establishes wire or wireless communication with external units via an antenna 29 or a connector 27. The control unit 22 is further connected to an internal power supply 24, such as a lithium ion capacitor, that supplies necessary electrical power to the functional units including the scan driver 15, the reader circuits 17, the storage unit 23, and the bias power supply 14. [Processes Carried out at Radiographic Image Capturing System during Capturing of Long-length image by One-Shot Exposure]

The processes carried out at the radiographic image capturing system 50 according to this embodiment during capturing of a long-length image by one-shot exposure will now be described in detail. The operation of the radiographic image capturing system 50 according to this embodiment will also be described.

Unlike a radiographic image capturing system 50 including a capturing room Ra and a console C connected to establish a 1:1 relationship, as illustrated in FIG. 1, a radiographic image capturing system 50 including multiple capturing rooms Ra (Ra1 to Ra3) connected to multiple consoles C via a network N, as illustrated in FIG. 2, requires the operator or radiologist to assign (declare) the capturing room Ra provided with the capturing stand 51A for capturing a long-length image by one-shot exposure with the console C to be used before image capturing. Once the capturing room Ra is assigned with the console C, the console C is linked to the assigned capturing room Ra.

The operator or radiologist starts the necessary radiographic image capturing devices 1 (i.e., turns on the power or switches from power saving mode to image capturing mode) and loads the started radiographic image capturing devices 1 in the holder 51a of the capturing stand 51A. That is, three radiographic image capturing devices 1 are loaded for capturing a long-length image of a full leg of an adult patient, and two for a young patient, i.e., a child or infant. The radiographic image capturing devices 1 carry out the initial operation including resetting of the radiation detectors 7.

### [Connecting Dongles to Radiographic Image Capturing Devices]

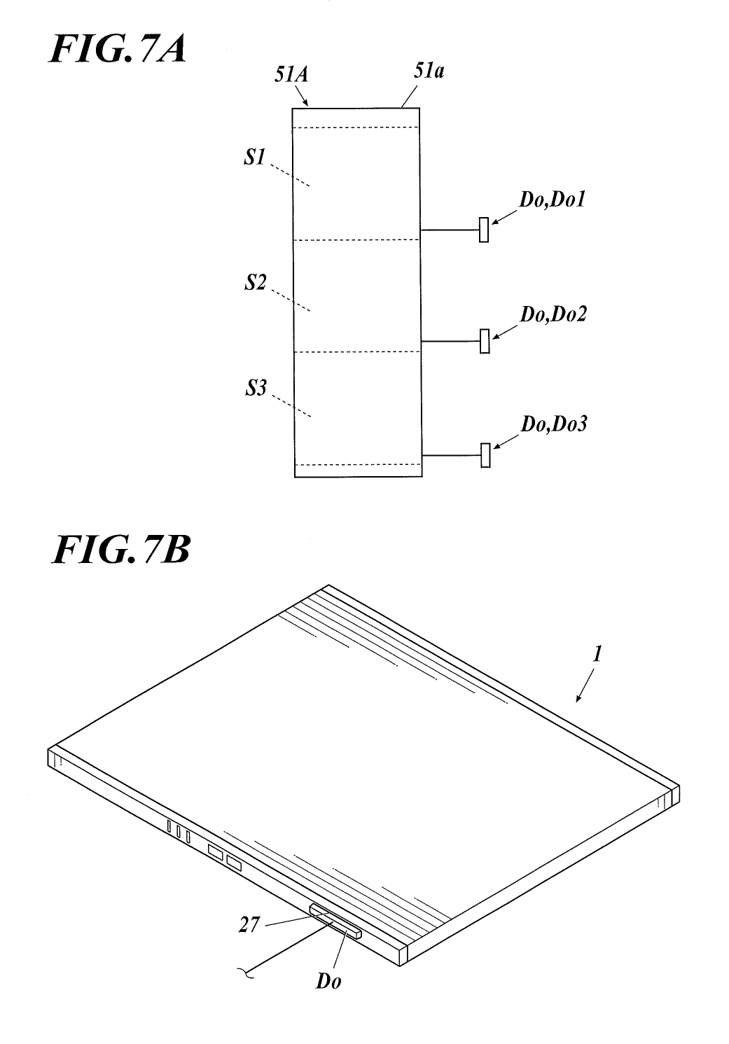

With reference to FIG. 7A, the holder 51a of the capturing stand 51A according to this embodiment is provided with dongles Do1 to Do3 that store respective identification information items and are respectively disposed at loading positions S1 to S3 in which the radiographic image capturing devices 1 can be loaded. With reference to FIG. 7B, a dongle Do is connected to the connector 27 of the radiographic image capturing device 1, and then the radiographic image capturing device 1 is loaded to the holder 51a.

Although not illustrated, the dongle Do may be connected to the radiographic image capturing device 1 via a USB terminal. Instead of manual connection of the dongle Do and the connector 27 of the radiographic image capturing device 1 by the operator or radiologist, the dongle Do corresponding to the loading position of the radiographic image capturing device 1 loaded in the holder 51a may be automatically connected to the connector 27 of the corresponding radiographic image capturing device 1.

Once the dongle Do is connected to the radiographic image capturing device 1, the radiographic image capturing device 1 reads the identification information stored in the dongle Do and sends this to the console C together with the identification information or cassette ID of the radiographic image capturing device 1. The console C stores a table showing the correspondence between the identification information items for the dongles Do1 to Do3 and the loading positions S1 to S3 in the holder 51a of the capturing stand 51A. Upon reception of the cassette ID and the identification information of the dongle Do from the radiographic image capturing device 1, the console C refers to the table and determines which loading position S1 or S3 in the holder 51a of the capturing stand 51A is loaded with the radiographic image capturing device 1.

The radiographic image capturing device 1 loaded in the holder 51a of the capturing stand 51A transmits, periodically or in response to a request from the console C, information on the remaining power in the internal power supply 24 (see FIG. 6) and the intensity of the wireless communication between the radiographic image capturing device 1 and the access point 53 (see FIGS. 1 and 2).

### [Capturing Order Information]

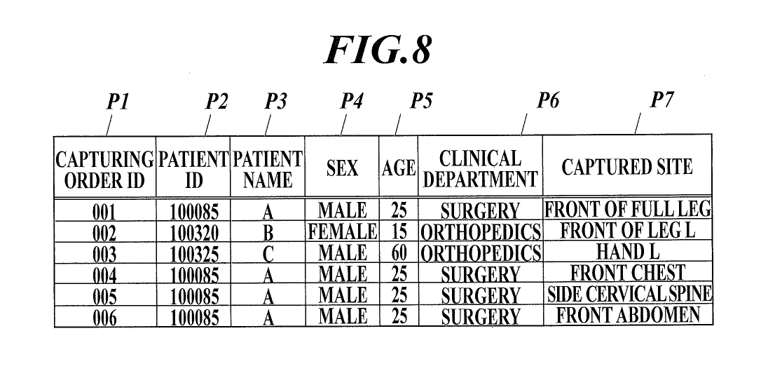

Prior to image capturing, the console C receives capturing order information on a scheduled radiographic image capturing from the HIS or the RIS in response to an operation by the operator or radiologist. With reference to the example illustrated in FIG. 8, the capturing order information according to this embodiment contains parameters of “patient ID” P2, “patient name” P3, “sex” P4, “age” P5, and “clinical department” P6, and “captured site” P7. “Capturing order IDs” P1 are automatically assigned to the capturing order information items in accordance with the order of registration of the capturing order.

In this embodiment, a capturing order information item containing a parameter corresponding to “full leg” or “full spine” for the captured site P7 involves capturing of a long-length image. The capturing order information may contain parameters for additional modalities (including the capturing stand 51A for capturing a long-length image by one-shot exposure). The parameters to be included in the capturing order information may be appropriately selected.

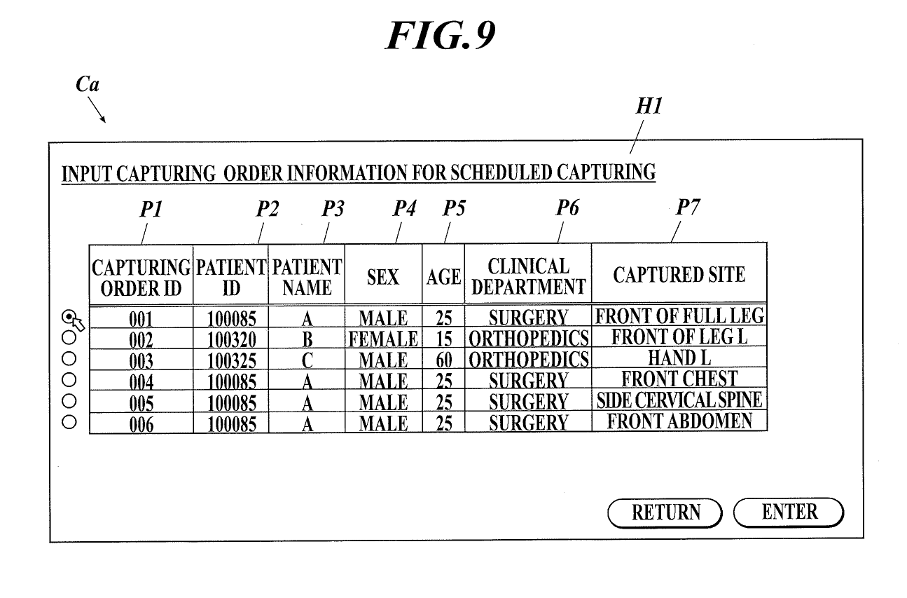

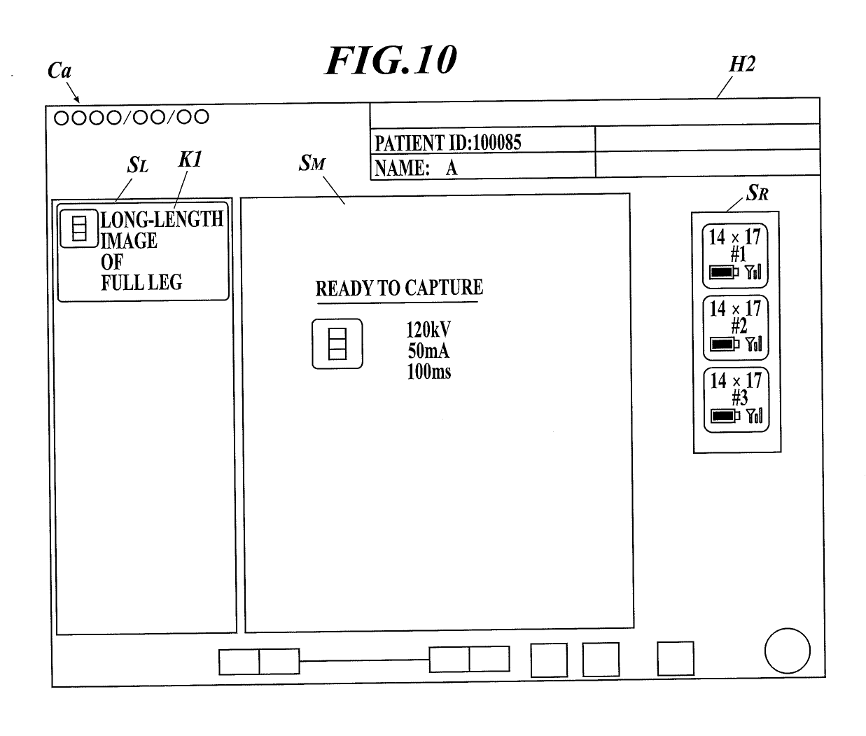

Upon reception of the capturing order information, the console C displays a selection menu H1 that contains a list of the capturing order information items on the display unit Ca, as illustrated in FIG. 9. The console C switches the selection menu H1 displayed on the display unit Ca to a menu screen H2, such as that illustrated in FIG. 10, in response to selection of a capturing order information item containing a parameter corresponding to “full front view of leg” (i.e., long-length image) for the captured site P7 on the selection menu H1.

### [Start of Radiographic Image Capturing Device]

As described above, upon selection of a capturing order information item on the console C, the console C may send necessary information to the controller 55 of the radiation irradiator 52 to automatically start the radiation irradiator 52. Alternatively, the operator or radiologist may manually operate the console 57 of the radiation irradiator 52 (see FIG. 1) to start the radiation irradiator 52.

### [Menu Screen]

In this embodiment, the menu screen H2 includes a main menu SM in the central area and sub-menus SL and SR respectively on the left and right of the main menu SM. The sub-menu SL in the left of the menu screen H2 displays a simplified icon of a capturing condition key K1 corresponding to the selected capturing order information item. The main menu SM in the central area of the menu screen H2 displays various items of information and an enlarged view of a generated image.

In this embodiment, the console C instructs the main menu SM. to display the phrase “Stand-by mode” or “Please wait” in a case where any one of the radiographic image capturing devices 1 loaded in the holder 51a of the capturing stand 51A is not ready for image capturing. With reference to FIG. 10, the console C instructs the main menu SM to display the phrase “Ready to capture” in a case where all of the radiographic image capturing devices 1 are available for image capturing. Such information may be announced by sound.

The console C instructs the main menu SM to display the phrase “Load panel” in a case where none of the radiographic image capturing devices 1 are loaded in the holder 51a of the capturing stand 51A (here “panel” refers to the radiographic image capturing device 1). In a case of battery exhaustion of the radiographic image capturing devices 1 or no wireless communication due to low signal strength determined based on the information on the remaining power in the internal power supplies 24 and the intensities of the wireless communication between the radiographic image capturing devices 1 and the access point 53 sent from the radiographic image capturing devices 1 in the holder 51a of the capturing stand 51A, as described above, the main menu SM displays the phrase “capturing not possible,” for example.

In the case when capturing cannot be performed, the loading position of the radiographic image capturing device 1 in the holder 51a that has an exhausted battery or is unable to establish wireless communication, among the multiple radiographic image capturing devices 1 loaded in the holder 51a of the capturing stand 51A, should be determined in order for the operator or radiologist to replace or switch the loading position of the relevant radiographic image capturing device 1.

With reference to FIG. 10, the console C according to this embodiment displays the sub-menu SR on the right of the menu screen H2 prior to image capturing to indicate information on the loading positions S1 to S3 of the radiographic image capturing devices 1 in the holder 51a of the capturing stand 51A, the remaining power in the internal power supplies 24, and the intensities of the wireless communication between the radiographic image capturing devices 1 and the access point 53.

The console C determines the loading positions S1 to S3 of the radiographic image capturing devices 1 in the holder 51a of the capturing stand 51A on the basis of information, such as the identification information, sent from the radiographic image capturing devices 1 connected to the holder 51a via the dongles Do (see FIGS. 7A and 7B).

The console C according to this embodiment displays the information on the remaining power in the internal power supplies 24 in the radiographic image capturing devices 1 and the intensities of the wireless communication between the radiographic image capturing devices 1 and the access point 53 in the form of vertically aligned, simplified icons corresponding to the loading positions S1 to S3 in the holder 51a, in the sub-menu SR.

[Notification of Available Capturing and Radiation from Radiographic Image Capturing Devices]

If the console C determines that all of the radiographic image capturing devices 1 loaded in the holder 51a of the capturing stand 51A are available for image capturing, the phrase “Capturing possible” is displayed on the main menu SM on the menu screen H2, as illustrated in FIG. 10, to notify the operator or radiologist that capturing of a long-length image by one-shot exposure can be performed.

The operator or radiologist positions the patient P as the subject and the holder 51a of the capturing stand 51A (i.e., the multiple radiographic image capturing devices 1) in the capturing room Ra (see FIGS. 1 and 2) and returns to the front chamber Rb to confirm the notification of available capturing displayed on the menu screen H2 of the console C and then operates the exposure switch 56 to emit radiation from the radiation irradiator 52.

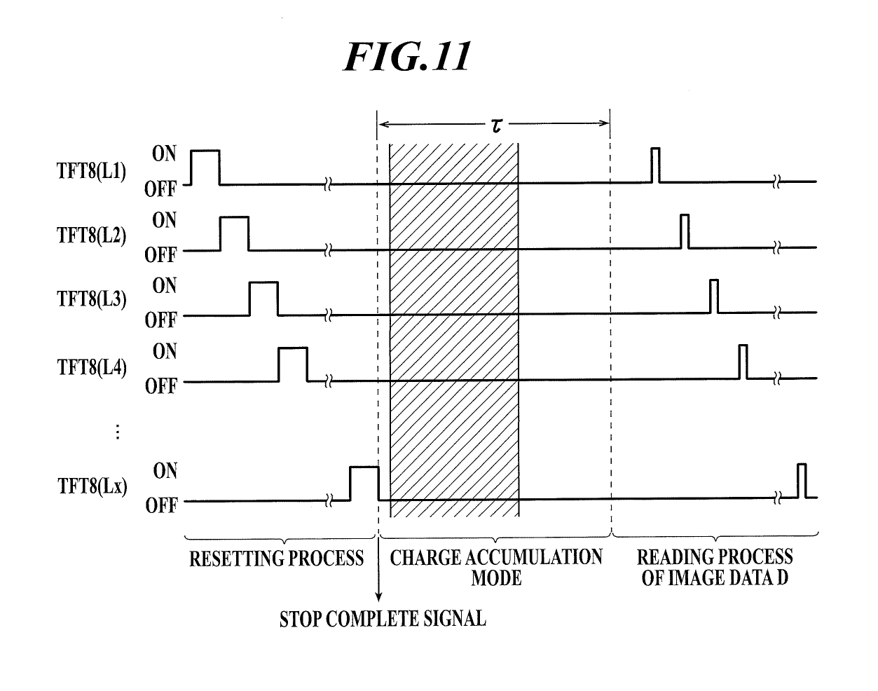

With reference to FIG. 11, each radiographic image capturing device 1 loaded in the holder 51a of the capturing stand 51A resets the radiation detectors 7 through sequential application of an ON voltage from the gate driver 15b of the scan driver 15 (see FIG. 6) to the lines L1 to Lx of the scanning lines 5, to neutralize the charges remaining in the radiation detectors 7.

In response to the operator or radiologist fully pressing the exposure switch 56, an irradiation start signal is sent from the controller 55 of the radiation irradiator 52 to the console C via the relay 54 or the access point 53. Upon reception of the irradiation start signal, the console C sends a signal instructing the stop of the resetting process of the radiation detectors 7 to the radiographic image capturing devices 1.

Each radiographic image capturing device 1 stops the ongoing process of resetting of the radiation detectors 7 immediately after application of an ON voltage to the last line Lx of the scanning lines 5. Upon stop of the resetting process of the radiation detectors 7, each radiographic image capturing device 1 sends a stop complete signal to the console C and applies an OFF voltage from the gate driver 15b to the lines L1 to Lx of the scanning lines 5, to enter a charge accumulation mode in which the charges generated in the radiation detectors 7 as a result of application of radiation are accumulated in the radiation detectors 7.

Upon reception of the stop completion signals from the radiographic image capturing devices 1 loaded in the holder 51a of the capturing stand 51A, the console C sends an interlock release signal to the controller 55 of the radiation irradiator 52. Upon reception of the interlock release signal, the controller 55 of the radiation irradiator 52 instructs the radiation irradiator 52 to emit radiation for the first time.

In this embodiment, a long-length image is captured by one-shot exposure through application of radiation from the radiation irradiator 52 to the radiographic image capturing devices 1 loaded in the holder 51a of the capturing stand 51A.

In this embodiment, the radiation irradiator 52 and the console C (and the radiographic image capturing devices 1) exchange signals with each other and operate in cooperation for capturing of a long-length image (coordinated image capturing), as described above. Alternatively, the radiation irradiator 52 and the radiographic image capturing devices 1 may not exchange signals for capturing of a long-length image (uncoordinated image capturing). In uncoordinated image capturing, the radiographic image capturing devices 1 detect the radiation from the radiation irradiator 52 and enter a charge accumulation mode. Schemes for the radiographic image capturing devices 1 to detect radiation are described in detail in, for example, Japanese Patent Application Laid-Open No. 2009-219538, WO2011/135917, and WO2011/152093.

### [Reading and Transmitting Image Data and Offset Data]

With reference to FIG. 11, each radiographic image capturing device 1 loaded in the holder 51a of the capturing stand 51A enters a charge accumulation mode, and radiation is emitted from the radiation irradiator 52 to capture a long-length image by one-shot exposure (the diagonally hatched area in FIG. 11 represents radiation emission). In response to this, the control unit 22 of each radiographic image capturing device 1 applies an ON voltage from the gate driver 15b to the line L1 to Lx of the scanning lines 5 to read the image data D described above.

The control unit 22 of each radiographic image capturing device 1 reads the image data D and wirelessly transmits the image data D to the console C via the antenna 29. That is, in a case where three radiographic image capturing devices 1 are loaded in the holder 51a of the capturing stand 51A, the three radiographic image capturing devices 1 simultaneously transmit the respective image data items D to the console C. Instead of first transmitting the image data D, preview image data Dp to be displayed on the menu screen H2, as described below, can be extracted from the image data D and transferred prior to the image data D.

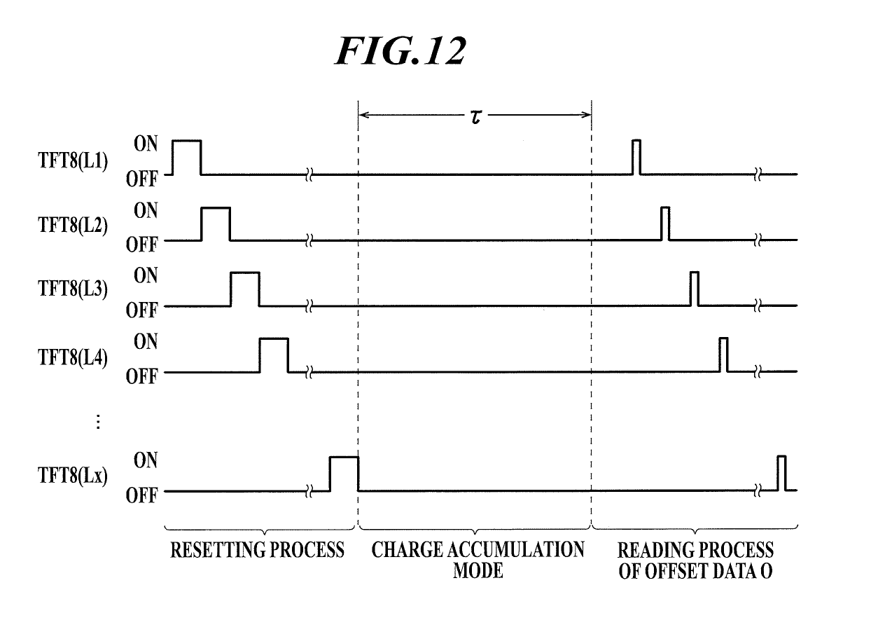

Simultaneous to the transfer of the image data D and the preview image data, the radiographic image capturing devices 1 read offset data O, as illustrated in FIG. 12. That is, the radiographic image capturing devices 1 read the image data D as described above and subsequently reset the radiation detectors 7 during one or more predetermined frames, as illustrated in the left of FIG. 12. The radiographic image capturing devices 1 then enter a charge accumulation mode.

The charge accumulation mode is continued during the time τ of the charge accumulation mode prior to the reading of the image data D, without irradiation of radiation to the radiographic image capturing devices 1. An ON voltage is then sequentially applied from the gate driver 15b to the line L1 to Lx of the scanning lines 5, as illustrated on the right of the FIG. 12, to read the offset data O from the radiation detectors 7 in a similar manner to the reading of the image data D described above. Alternatively, the reading of the offset data O may be carried out before the capturing of a long-length image.

After reading the offset data O, each radiographic image capturing device 1 transfers the remaining image data D and the offset data O to the console C if the preview image data Dp has already been transferred. If the preview image data Dp has not been extracted, each radiographic image capturing device 1 completes the transfer of the image data D that has already been started and subsequently transfers the offset data O to the console C.

### [Display of Preview Image]

Every time the image data D or the preview image data Dp is transferred from the radiographic image capturing devices 1 to the console C (hereinafter, the image data D will be representatively described, but the same description holds for the preview image data Dp), the console C calculates the difference Dp* defined by expression (1) for each radiation detector 7 for each radiographic image capturing device 1:

Dp*=D−Op (1)

where D is the image data D, and Op is assumed offset data O provided because the offset data O is not yet received.

The console C displays a preview image p_pre at positions corresponding to the relevant radiographic image capturing device 1 on the sub-menu SR on the right of the menu screen H2 each time the console C carries out simple image processing on the value Dp*. As a result, the preview images p_pre are displayed as wipe images at positions corresponding to the respective radiographic image capturing devices 1 in the sub-menu SR on the right of the menu screen H2 (i.e., the images are displayed by overwriting the display area from top to bottom, for example).

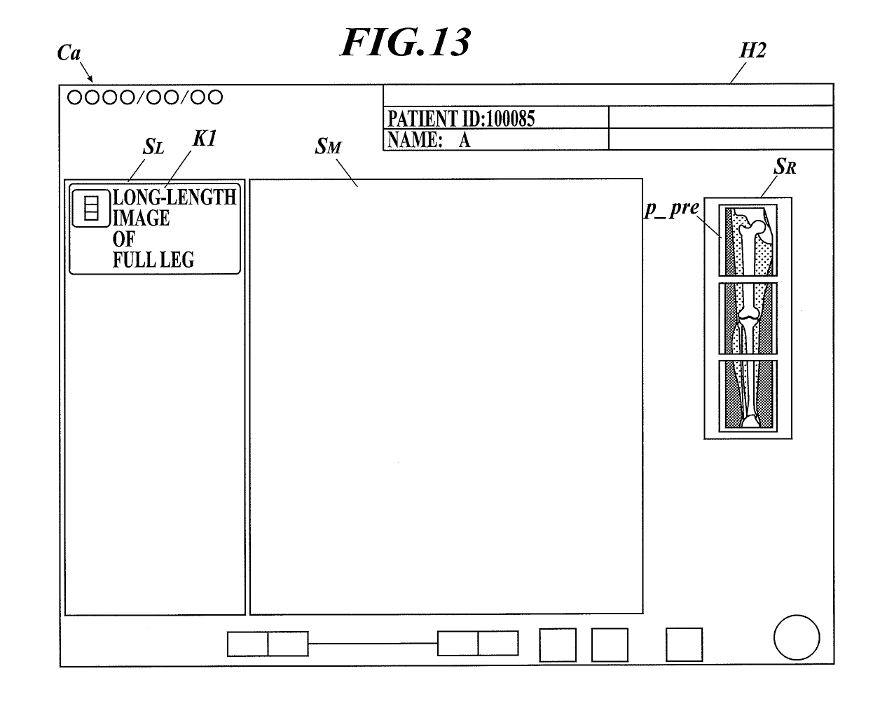

Upon completion of the transmission of the image data D from the radiographic image capturing devices 1, the preview images p_pre are displayed at the positions corresponding to the respective radiographic image capturing devices 1 in the sub-menu SR on the right of the menu screen H2, as illustrated in FIG. 13, for example. The operator or radiologist observes these preview images p_pre and determines the necessity of recapturing.

### [Overview of Image Generation and Long-Length Image Generation]

Upon reception of the image data D and the offset data O from the radiographic image capturing devices 1, the console C calculates the true image data D* by subtracting the offset data O from the image data D for each radiation detector 7 in each radiographic image capturing device 1, as defined by expression (2):

D*=D−O (2)

The console C generates images p on the basis of the calculated true image data sets D* for the radiographic image capturing devices 1 and combines the images p to generate a long-length image “plong.” The overview of the generation of the images p for the radiographic image capturing devices 1 and the long-length image “plong” will now be described.

Various schemes may be applied to generate the images p and the long-length image “plong.” A typical example will now be described. In the following example, three radiographic image capturing devices 1 are loaded in the holder 51a of the capturing stand 51A, and a long-length image of the right leg of an adult is captured. Hereinafter, the true image data D* is simply referred to as image data D*. The image data sets D* and the images p corresponding to the three radiographic image capturing devices 1 are referred to as D*1, D*2, and D*3, and p1, p2, and p3, respectively, from the side closer to the head of the patient (i.e., from the side farther from the toe).

The console C calculates the image data sets D*1 to D*3 for each radiographic image capturing device 1 as described above and then confirms the area containing the subject (i.e., bone and organs of the patient) in temporary images p*1 to p*3 (not shown) corresponding to two-dimensionally arranged image data sets D*1 to D*3. The console C then assigns regions of interest (ROI) in the respective temporary images p*1 to p*3. It is well known that an ROI is assigned through automatic extraction of a specific anatomical structure of the human body for each image or assigned to a predetermined area in the images, for example.

The console C deletes any abnormal values of the image data sets D*1 to D*3 (i.e., signal values) of the pixels (i.e., radiation detectors 7) in the ROI for the temporary images p*1 to p*3 and creates a histogram to determine the distributions of the image data sets D*1 to D*3. The console C then normalizes the image data sets D*1 to D*3 (normalization) to correct the variation in the distributions of the image data sets D*1 to D*3 due to a variation in the irradiation conditions attributed to the body shape of the patient.

In the normalization process, the image data set D*1 acquired as described above is adjusted such that the maximum value DH(1) and the minimum value DL(1) in the distribution of the image data set D*1 equal a predetermined maximum value SH and a predetermined minimum value SL, respectively. That is, normalization is achieved by adjusting the image data D*1 such that the range of the image data set D*1 (DH(1) to DL(1)) equals the range of the maximum value SH to the minimum value SL.

Specifically, the console C determines S(1) and G(1) for converting the image data set D*1 to the normalized data set D**1 by expression (3):

D**1=G(1)×D*1+S(1) (3)

where,

G(1)=(SH−SL)/(DH(1)−DL(1)) (4)

S(1)=(SL·DH(1)−SH·DL(1))/(DH(1)−DL(1)) (5)

In the above expressions, G represents a contrast value (slope), and S represents a concentration correction value (intercept). The console C also normalizes the distributions of the image data sets D*2 and D*3 through a similar process to determine G(2), S(2), G(3), and S(3) and converts the image data sets D*2 and D*3 to normalized data sets D**2 and D**3, respectively.

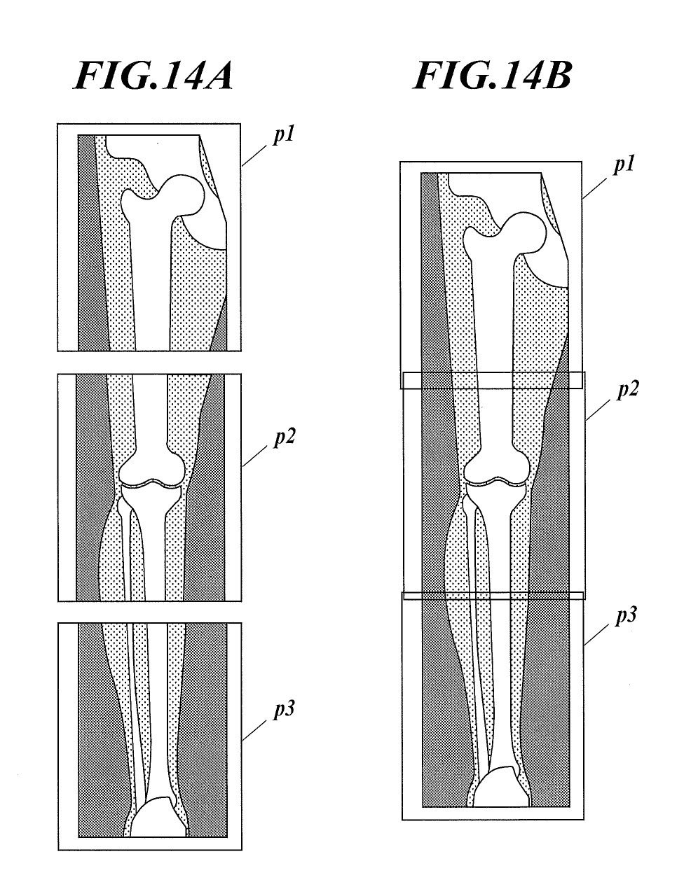

The console C then carries out image processing such as gradation processing on the image data set D*1 normalized as described above (i.e., the normalized data set D**1) with reference to a lookup table (LUT) corresponding to the captured site (full leg or full spine, for example), to generate images p1 to p3 for the respective radiographic image capturing devices 1, as illustrated in FIG. 14A.

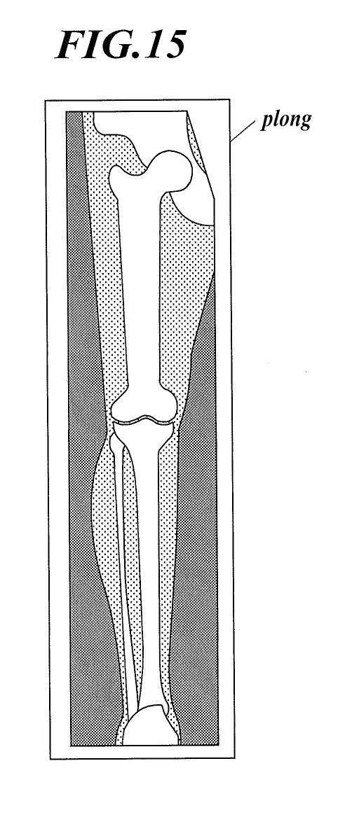

The console C aligns the edges of the images p1 to p3, as illustrated in FIG. 14B, and combines the images p1 to p3 to generate a long-length image “plong,” as illustrated in FIG. 15. A known scheme, such as that described in Japanese Patent Application Laid-Open No. 2013-154146, may be applied for alignment and combination of the images p1 to p3.

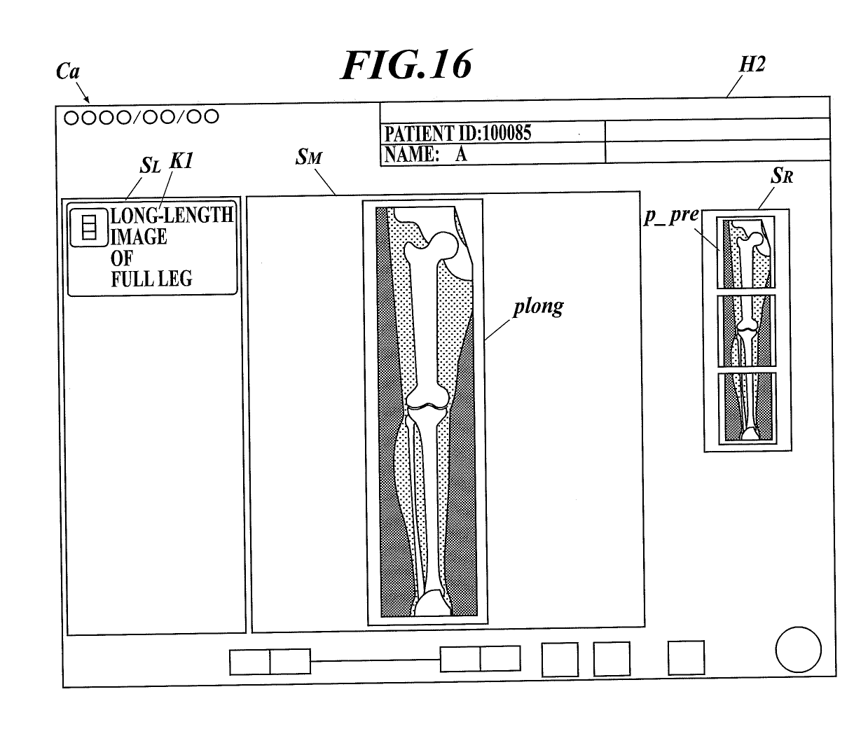

With reference to FIG. 16, the console C displays the generated long-length image “plong” in the main menu SM on the menu screen H2. The displayed long-length image “plong” is output to an external system, such as PACS, through selection by the operator or radiologist or linking to a capturing condition key K1, for example.

### [Problems in Conventional Image Processing of Long-Length Image Captured by One-Shot Exposure]

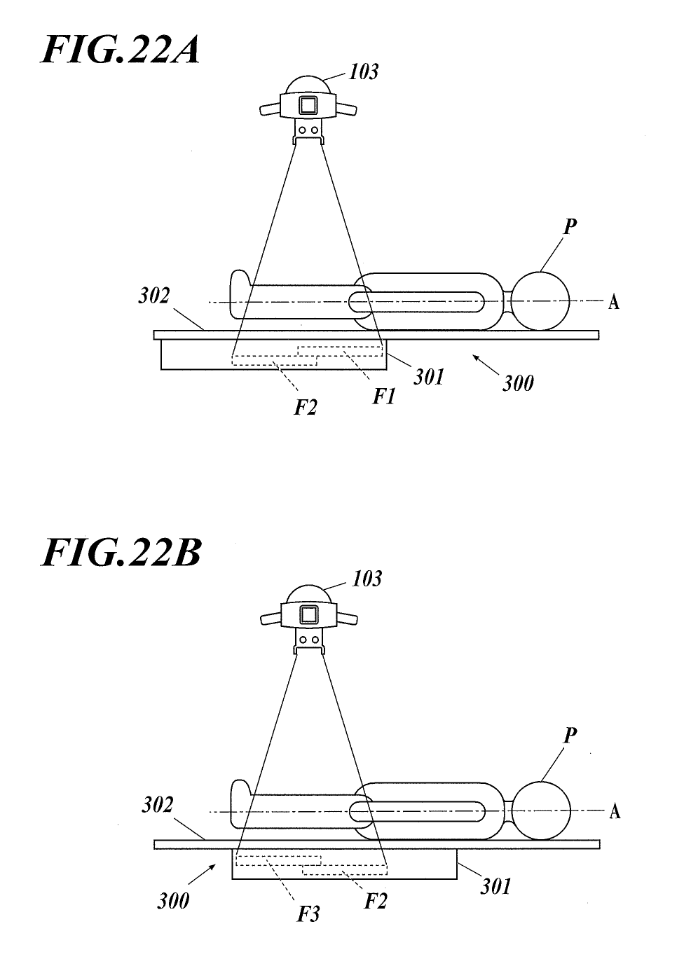

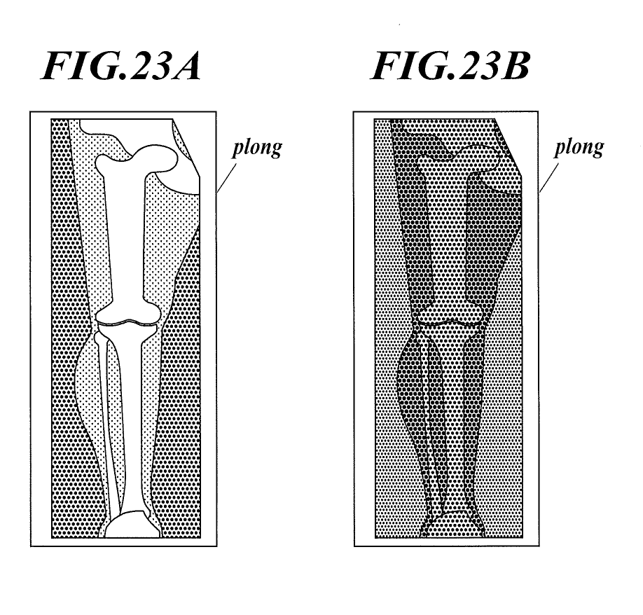

A long-length image “plong” of a full front view of a leg of a young patient P, i.e., a child or infant, is generated through combination of images p1 to p3, which are normalized as described above and captured with two radiographic image capturing devices 1 loaded in the holder 51a of the capturing stand 51A for capturing a long-length image by one-shot exposure, which holds a total of three radiographic image capturing devices 1, as illustrated in FIGS. 22A and 22B (i.e., radiographic image capturing devices F1 and F2 in FIG. 22A, and radiographic image capturing devices F2 and F3 in FIG. 22B). The long-length image “plong” acquired in this way may have varying overall brightness and contrast, as illustrated in FIGS. 23A and 23B.

The inventors conducted a research on this problem and discovered the following cause of the problem.

In the normalization process of the long-length image, the image data sets D*1 to D*3 are normalized to respectively acquire normalized data sets D**1 to D**3, and then one of the image data sets D* of the image data sets D*1 to D*3 is selected as a reference for matching the image quality of the normalized data sets D**1 to D**3, i.e., the contrast values G and the concentration correction values S. The contrast value G and the concentration correction value S used for the normalization of the reference image data set D* are applied to the other image data sets D* for conversion. This holds for both the image processing according to this embodiment and conventional image processing for capturing a long-length image by one-shot exposure.

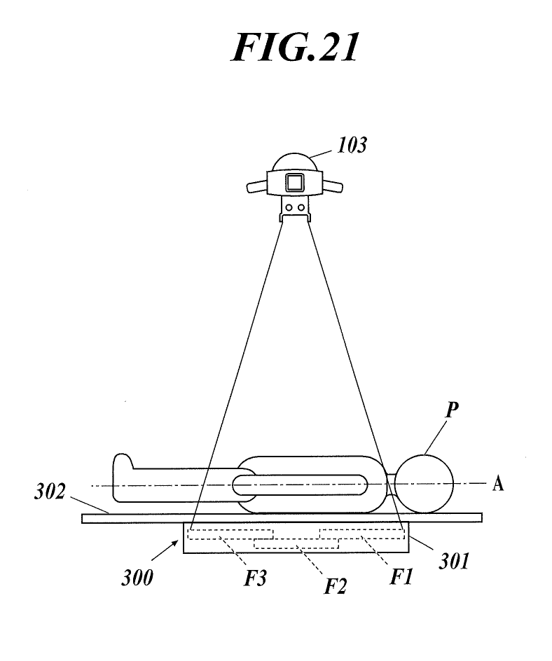

In the image processing for a conventional long-length image captured by one-shot exposure, the image data set D*2 is selected as the reference image data set D* because a radiographic image capturing device F is always loaded in the position of the radiographic image capturing device F2 in FIG. 21 (corresponding to the loading position S2 in the holder 51a of the capturing stand 51A according to this embodiment, as illustrated in FIG. 7A) whether three radiographic image capturing devices F1 to F3 (see FIG. 21), two radiographic image capturing devices F1 and F2 (see FIG. 22A), or two radiographic image capturing devices F2 and F3 (see FIG. 22B) are loaded in the holder 301 of the capturing stand 300.

The contrast value G(2) and the concentration correction value S(2) used for the normalization of the reference image data set D*2 is applied to the other image data sets D*1 and D*3 so as to determine converted data sets D***1 and D***3 by expressions (6) and (7):

D***1=G(2)×D*1+S(2) (6)

D***3=G(2)×D*3+S(2) (7)

Image processing, such as gradation processing, with reference to LUTs corresponding to the captured sites is carried out on the normalized data set D**2, which is acquired through normalization of the reference image data set D*2, and converted data sets D***1 and D***3 respectively acquired from the image data sets D*1 and D*3, as described above, to generate the images p1 to p3, which are combined to generate a long-length image “plong.”

The image data set D*2 acquired from the reference radiographic image capturing device F2 of the two radiographic image capturing devices F1 and F2 loaded in the holder 301 of the capturing stand 300, as illustrated in FIG. 22A, contains an image of the knee, the tibia, and the fibula of the leg. A region of interest (ROI) corresponding to these features is assigned, and the distribution of the image data set D*2 for the pixels in the ROI is determined. The contrast value G(2) and the concentration correction value S(2) are determined on the basis of this distribution.

The image data set D*2 acquired by the reference radiographic image capturing device F2 of the two radiographic image capturing devices F2 and F3 loaded in the holder 301 of the capturing stand 300, as illustrated in FIG. 22B, capture the pelvis and femur of the full leg. An ROI corresponding to these features is assigned, and the distribution of the image data set D*2 for the pixels in the ROI is determined. The contrast value G(2) and the concentration correction value S(2) are determined on the basis of this distribution.

The contrast value G(2) and the concentration correction value S(2), which are used for normalization, differ between the conventional radiographic image capturing devices F loaded as illustrated in FIG. 22A and those loaded as illustrated in FIG. 22B. Thus, the same processes of capturing front images of a full front view of a leg of a young patient P, i.e., an infant, generate long-length images “plong” having varying image qualities (i.e., the overall brightness (associated with the concentration correction value S(2)) and the contrast (associated with the contrast value G(2))), as illustrated in FIGS. 23A and 23B.

The radiographic image capturing system 50 according to this embodiment selects a radiographic image capturing device 1 among the radiographic image capturing devices 1 loaded in a predetermined order in the holder 51a of the capturing stand 51A during image capturing as a reference for image processing including normalization, instead of selecting a conventional radiographic image capturing device F at a predetermined loading position (the loading position S2 in the example described above) in the holder of the capturing stand.

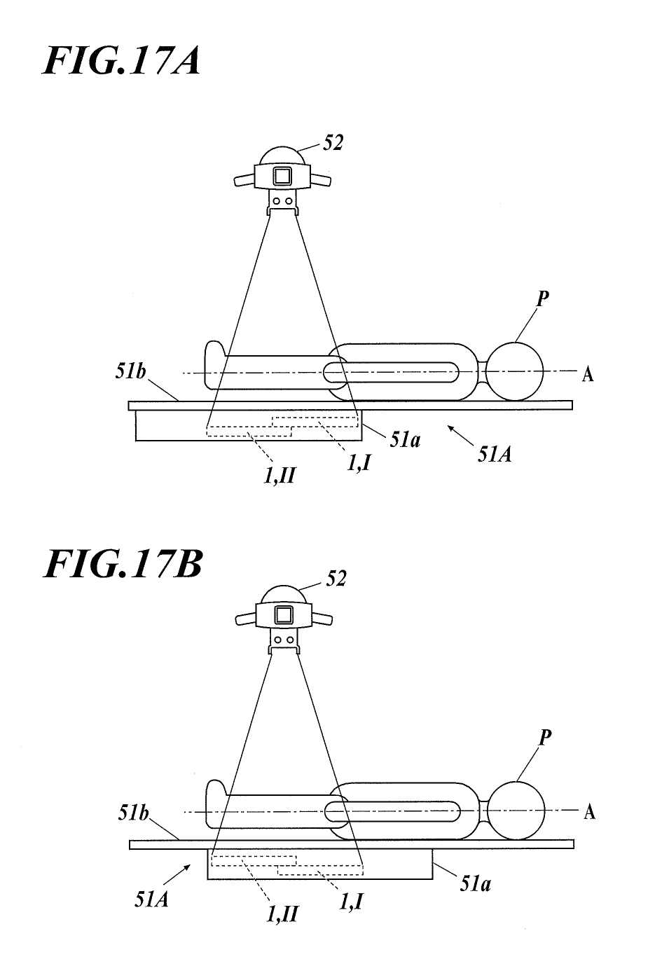

In this embodiment, the holder 51a of the capturing stand 51A holds a maximum of three radiographic image capturing devices 1. If two radiographic image capturing devices 1 are loaded in the holder 51a, i.e., the number of loaded radiographic image capturing devices 1 is less than the number of loading positions in the holder 51a, and if the radiographic image capturing devices 1 are irradiated with radiation from the radiation irradiator 52 to capture a long-length image by one-shot exposure, as illustrated in FIGS. 17A and 17B, the console C detects the two radiographic image capturing devices 1 loaded in the holder 51a of the capturing stand 51A on the basis of identification data items on the dongles Do sent from the respective radiographic image capturing devices 1.

FIG. 17A illustrates the capturing of a long-length image of a full front view of a leg with two radiographic image capturing devices 1 loaded at two loading positions closer to the head of the patient among the loading positions in the holder 51a of the capturing stand 51A. FIG. 17B illustrates the capturing of a long-length image of a full front view of a leg with two radiographic image capturing devices 1 loaded at two loading positions closer to the toe of the patient among the loading positions.

The console C carries out image processing including normalization by applying parameters, i.e., the contrast value G and the concentration correction value S described above, to image data D* acquired, through application of radiation from the radiation irradiator 52, from one of the two radiographic image capturing devices 1 loaded in the holder 51a assigned with a predetermined number counted from the head to the toe of the patient P as the subject (see FIGS. 17A and 17B in which the radiographic image capturing devices 1 are numbered “I” and “II”). The same parameters, i.e., the contrast value G and the concentration correction value S, are also applied to image data D* acquired from the other radiographic image capturing device 1 for image processing.

Specifically, with reference to FIGS. 17A and 17B, if, for example, the predetermined number is “II,” the console C assigns a region of interest (ROI) to the image data D*II acquired, through application of radiation from the radiation irradiator 52, from the radiographic image capturing device 1(II) assigned with the number “II” of the two radiographic image capturing devices 1 loaded in the holder 51a and numbered “I” and “II” in an ascending order from the head to the toe of the patient P as the subject. The console C calculates the contrast value G(II) and the concentration correction value S(II) and normalizes the image data D*II such that the maximum value DH(II) and the minimum value DL(II) equal a predetermined maximum value SH and a predetermined minimum value SL, respectively, to calculate the normalized data D** (II) by expressions (4) and (5).

The contrast value G(II) and the concentration correction value S(II) used for the normalization of the reference image data D*II is also applied to the image data D*I acquired from the other radiographic image capturing device 1(I), to calculate the converted data D***I by the expressions (6) and (7). In this embodiment, image processing is carried out on the image data sets D* as described above.

Image processing including gradation processing is carried out with reference to an LUT for the captured site of the normalized data D**II acquired through normalization of the reference image data D*II and the converted data D***I acquired through conversion of the image data D*I, to generate images pI and pII (not shown). The images pI and pII are combined to generate the long-length image “plong.” The same description also holds for the predetermined number “I.”

With this configuration, the image data D*II acquired by the reference radiographic image capturing device 1(II) assigned with the number “II” always captures the knee, the tibia, and the fibula of the leg, regardless of the loading positions of the two radiographic image capturing devices 1(I) and 1(II), as illustrated in FIG. 17A or 17B. The image data D*II is assigned to a region of interest (ROI) corresponding to an area including the knee, the tibia, and the fibula, with the two radiographic image capturing devices 1 loaded at the positions illustrated in either FIG. 17A or 17B. The distribution of the image data D*II for the pixels in the ROI is determined, and the contrast value G(II) and the concentration correction value S(II) are determined based on this distribution.

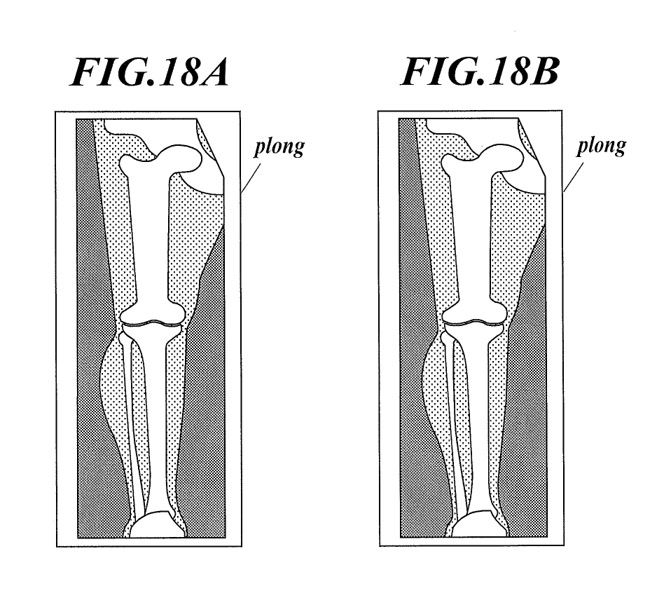

The contrast value G(II) and the concentration correction value S(II) determined with the two radiographic image capturing devices 1 loaded at the positions illustrated in FIG. 17A are exactly or substantially equal to those determined with the two radiographic image capturing devices 1 loaded at the positions illustrated in FIG. 17B. Thus, the long-length image “plong” generated from the former (see FIG. 18A) and the long-length image “plong” generated from the latter (see FIG. 18B) have the same image quality (i.e., overall brightness and contrast).

According to this embodiment, long-length images “plong” having exactly or substantially the same image qualities (i.e., overall brightness and contrast) can be generated through capturing of a long-length image with the multiple radiographic image capturing devices 1 loaded at any of the loading positions in the holder 51a of the capturing stand 51A, the number (two in the example above) of the radiographic image capturing devices 1 loaded in the holder 51a being smaller than the maximum number (three in the example above) of radiographic image capturing devices 1 loadable in the holder 51a.

### Advantageous Effects

As described above, a long-length image can be captured by one-shot exposure with the multiple radiographic image capturing devices 1 loaded at the loading positions in the holder 51a of the capturing stand 51A, the number (two in the example above) of the radiographic image capturing devices 1 loaded in the holder 51a being smaller than the maximum. number (three in the example above) of radiographic image capturing devices 1 loadable in the holder 51a. In this way, the radiographic image capturing system 50 according to this embodiment can generate long-length images “plong” having the same image quality, regardless of the loading positions of the multiple radiographic image capturing devices 1 in the holder 51a of the capturing stand 51A, unlike a conventional system that generates long-length images “plong” having different qualities depending on the loading positions of the radiographic image capturing devices F in the holder.

Thus, a medical doctor, for example, can observe multiple long-length images “plong” and readily and accurately compare the long-length images “plong” having similar image qualities, to provide an appropriate pathological diagnosis and appropriate treatment.

In this embodiment, the predetermined number is “II.” Alternatively, the predetermined number may be “I,” as described above. In such a case, the image data D*I captured by the radiographic image capturing device 1(I) assigned with the number “I” and disposed at the loading position illustrated in either FIG. 17A or 17B contains the pelvis and the femur of the leg. Thus, the contrast value G(I) and the concentration correction value S(I) calculated with the two radiographic image capturing devices 1 loaded at the positions illustrated in FIG. 17A are the exactly or substantially equal to those calculated with the radiographic image capturing devices 1 loaded at the positions illustrated in FIG. 17B.

In this case also, the long-length images “plong” generated from the former and the long-length images “plong” generated from the latter have similar image qualities (i.e., overall brightness and contrast), as illustrated in FIGS. 18A and 18B.

In this embodiment, long-length images are captured with the radiographic image capturing devices 1 loaded at the loading positions in the holder 51a of the capturing stand 51A, the number of the radiographic image capturing devices 1 loaded in the holder 51a being smaller than the maximum number of radiographic image capturing devices 1 loadable in the holder 51a. Alternatively, the present invention may be applied to the capturing of a long-length image with the radiographic image capturing devices 1 with the maximum number of radiographic image capturing devices 1 loaded in the holder 51a (i.e., three radiographic image capturing devices 1 loaded in the holder 51a that can carry three radiographic image capturing devices 1). This case also has the same advantageous effects as those described above.

In this embodiment, the contrast value G and the concentration correction value S are parameters to be applied to the reference image (assigned with a predetermined number) and the images other than the reference image during image processing. Alternatively, in the present invention, LUTs and parameters, such as correction values, may be applied to the reference image and the images other than the reference image. [Application to Preview Image]

The image processing scheme described above, i.e., image processing carried out by applying the parameters (e.g., the contrast value G and the concentration correction value S) applied to the reference image data set D* to other image data sets D*, can also be applied to the display of preview images p_pre (see FIG. 13), for example. With reference to FIG. 13, the display of preview images p_pre will now be described for a case of three radiographic image capturing devices 1 loaded in the holder 51a of the capturing stand 51A, three being the maximum number of radiographic image capturing devices 1 loadable in the holder 51a. As described above, the same description holds for a case of two radiographic image capturing devices 1 loaded in the holder 51a of the capturing stand 51A, two being a number smaller than the maximum number.

As described above in this embodiment, every time the console C receives image data D or preview image data Dp (hereinafter, the image data D will be representatively described, but the same description holds for the preview image data Dp) from the radiographic image capturing devices 1, the console C calculates the difference Dp* of the image data D and the assumed offset data Op defined by the expression (1) for each radiation detector 7 in each radiographic image capturing device 1 and displays a wipe image of each preview images p_pre in the sub-menu SR on the right of the menu screen H2.

A region of interest (ROI) can be assigned to each preview image p_pre, as described above, after 80% to 90% of the preview images p_pre is displayed. Once the ROI is assigned, any abnormal values among the values Dp* of the pixels in the ROI are deleted from each preview image p_pre, and a histogram is created to determine the distributions of the values Dp*. As described above, the contrast value G and the concentration correction value S are calculated, and normalization is carried out to generate the normalized data Dp**.

As described above, the contrast value G and the concentration correction value S used for the normalization of the reference preview image p_pre (assigned with the number “II,” for example) are applied to the other preview images p_pre for conversion to converted data Dp***. Simple image processing is carried out on the normalized data D** acquired through normalization of the reference image data set D* and the converted data set D*** acquired through conversion of the other image data sets D*, as described above, to normalize and convert the preview images p_pre for displaying wipe images of the preview images p_pre.

This reduces the variation in image quality of the preview images p_pre, which are displayed as wipe images on the sub-menu SR on the right of the menu screen H2. The images have similar qualities regardless of the loading positions of the multiple radiographic image capturing devices 1 in the holder 51a of the capturing stand 51A, unlike the images captured with conventional radiographic image capturing devices F, which have varying brightness depending on the loading position.

Unlike the images generated with the radiographic image capturing devices F loaded in the holder of the capturing stand, which are dark and difficult to observe depending on the loading positions of the radiographic image capturing devices F, the preview images p_pre displayed as wipe images in the sub-menu SR on the right of the menu screen H2 have similar image qualities regardless of the loading positions of the radiographic image capturing devices 1 in the holder 51a of the capturing stand 51A. Thus, the operator or radiologist can observe the images and accurately determine the necessity of recapturing.

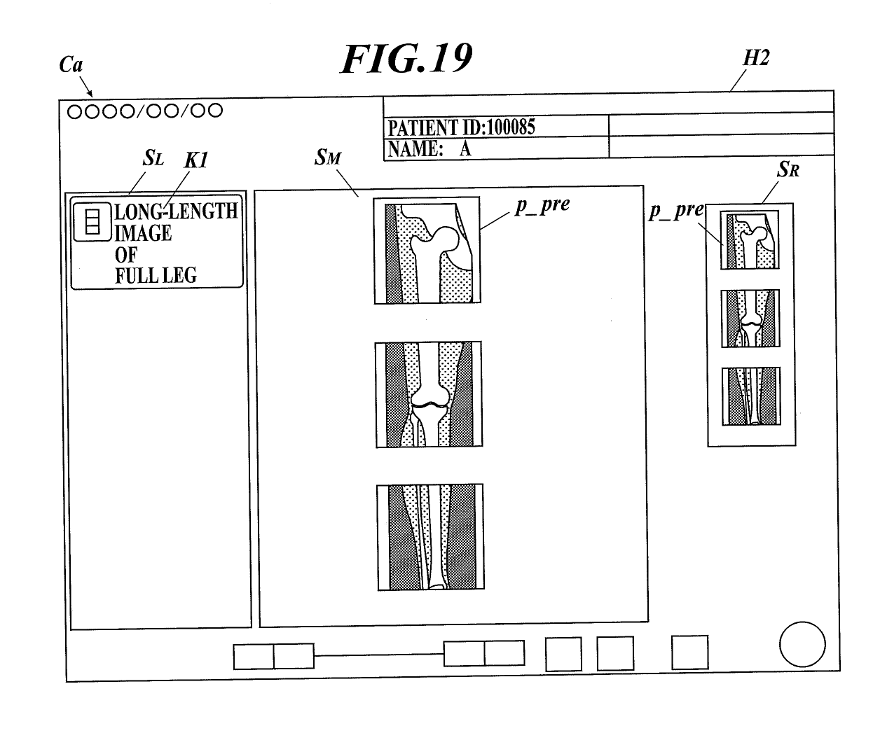

With reference to FIG. 19, enlarged views of the preview images p_pre normalized and converted as described above may be displayed (as wipe images) in the main menu SM on the menu screen H2. This allows the operator or radiologist to readily observe the preview images p_pre and accurately determine the necessity of recapturing. [Output of Images to External System]

In this embodiment, image processing is carried out on the image data D* acquired by the radiographic image capturing devices 1 loaded in the holder 51a of the capturing stand 51A, to generate images p1 to p3, as described above. The images p1 to p3 are combined to generate a long-length image “plong” (see FIGS. 15 and 16).

Alternatively, the console C in a medical facility, for example, may carry out the image process to generate the images p1 to p3, as described above. The images p1 to p3 then may be output to an external system, such as the QA station, mentioned above. The external system may combine the images p1 to p3 to generate the long-length image “plong.” In this case also, the present invention can be applied to generate the images p1 to p3 through image processing carried out by the console C, as described above, and then send the generated images p1 to p3 to the external system.

Instead of capturing images of the head, chest, or abdomen of a patient who has been transported by an ambulance, for example, through application of radiation from the radiation irradiator 52, a long-length image can be captured through one-shot exposure of the capturing stand 51A (i.e., radiation is emitted only once from the radiation irradiator 52). This reduces the time required for image capturing. Thus, the patient can quickly start other treatments.

If the images of the head, chest, and abdomen have varying qualities, ready diagnosis by a medical doctor could be difficult.

Thus, in such a case, image processing is carried out by applying parameters that are applied to the reference image data D*, such as the contrast value G and the concentration correction value S, to the image data sets D* acquired by the radiographic image capturing devices 1 as described above, so as to match the image qualities of the images p1 to p3. The images are output to an external system, such as PACS. A medical doctor can download these images from the PACS to a reader and observe the images to provide an accurate diagnosis.

The present invention is not limited to the above embodiments and modifications, and can be suitably changed without leaving the scope of the present invention.

This application is based upon and claims the benefit of priority from the Japanese Patent Application No. 2015-78113, filed Apr. 7, 2015, the entire contents of which are incorporated herein by reference.

What is claimed is:

1. A radiographic image capturing system comprising:
a capturing stand including a holder which can hold a plurality of radiographic image capturing devices;
a radiation irradiator that is able to apply radiation to the radiographic image capturing devices loaded in the holder at once; and
a console that carries out image processing on image data acquired by the radiographic image capturing devices,
wherein, the console carries out the image processing during capturing of a long-length image acquired by the radiographic image capturing devices loaded in the holder of the capturing stand through application of a parameter applied to image data acquired by a radiographic image capturing device assigned with a predetermined number to image data acquired by the other radiographic image capturing devices, the predetermined number being a number from multiple numbers assigned to the radiographic image capturing devices loaded in the holder during the application of radiation from the radiation irradiator in an ascending order from a head to toe of a patient as a subject, a number of the radiographic image capturing devices loaded in the holder being smaller or equal to a maximum number of radiographic image capturing devices loadable in the holder.

2. The radiographic image capturing system according to claim 1, wherein, the console generates a long-length image by combining images generated through the image processing carried out on the image data acquired from each radiographic image capturing device.

3. The radiographic image capturing system according to claim 1, wherein, the console outputs the images generated through image processing carried out on the image data acquired from each radiographic image capturing device to an external system.
